(19) United States
(12) Patent Application Publication
(10) Pub. No.: US 2010/0004582 A1
(43) Pub. Date: Jan. 7, 2010

(54) Wide-Area Parasystemic Treatment of Skin Related Conditions

(75) Inventors: Thomas Bragagna, Feldkirch (AT); Christof Böhler, Berneck (CH); Nicolas Zech, Zurich (CH)

Correspondence Address: FISH & ASSOCIATES, PC;ROBERT D. FISH, 2603 Main Street, Suite 1000, Irvine, CA, 92614-6232, US

(73) Assignee: PANTEC BIOSOLUTIONS AG, Ruggell (LI)

(21) Appl. No.: 12/447,030

(22) PCT Filed: Oct. 25, 2007
(86) PCT No.: PCT/EP2007/061500

§ 371 Date: Apr. 24, 2009

(30) Foreign Application Priority Data
Oct. 25, 2006 (EP) PCT/EP2006/067772

(51) Int. Cl. A61M 5/00

(52) U.S. Cl. 604/20

(57) ABSTRACT

Use of an immunomodulatory, immunostimulatory, or immunosuppressive drug in the manufacture of a medicament for treatment of a skin related disease or disorder, wherein the drug is formulated for topical administration to porated skin having a plurality of pores with predetermined geometry such that the concentration of the drug in combination with the predetermined geometry of the pores are effective in treatment of the skin related disease or disorder. The method of facilitating treatment of a skin related disease or disorder, comprising providing information that a drug is effective in treatment of the skin related disease or disorder; providing information to apply the drug to an area of porated skin comprising a plurality of pores; wherein the information specifies that the area is equal or greater than 1 cm²; wherein the information further specifies that at least some of the plurality of pores have a predetermined geometry; and wherein the predetermined geometry is effective to substantially prevent systemic administration of the drug.

### FIELD OF THE INVENTION

This invention relates to an improved method and kit to facilitating treatment of a skin related disease or disorder. The invention further relates to the use of a drug in the manufacture of a medicament for treatment of a skin related disease or disorder.

### BACKGROUND OF THE INVENTION

Numerous diseases and conditions involve skin in a direct or indirect manner, and most of the diseases and conditions are associated with or caused by an immunologic response to an exogenous stimulus. While an immunologic response is generally desirable in most instances (e.g., to combat infection), an auto- or alloimmune response is typically detrimental (e.g., in skin transplantation). Treatment of many skin diseases and conditions is often local and topical in response to an etiologic agent or stimulus (e.g., injury or infection). However, various other skin diseases and conditions are more diffuse in presentation and may be the result of regional agents or stimuli (e.g., exposure to allergen, radiation, etc.) and may in some cases even have systemic underlying conditions (e.g., histoincompatibility).

While topical treatment is often relatively simple and effective in many cases, treatment of diseases and conditions that manifest themselves in a relatively large area or have a systemic component is significantly more difficult. Conceptually, two treatment options are available.

First, a drug may be applied over a relatively large-area (e.g., by application of topical ointment), but efficacy is often undesirable as the skin presents a permeability barrier to most compounds with molecular weight greater 500 Dalton. Even when the drug is a relatively small molecule, hydrophilic drugs will typically not be effectively delivered. Several strategies have been developed to circumvent at least some of these problems, including chemical approaches (e.g., micro/nanovesicle delivery via encapsulation into liposome and other lipophilic carriers; penetration enhancers, such as azone, alphahydroxy acids, etc) and mechanical approaches (e.g., partial removal of stratum corneum by tape stripping, dermabrasion, etc.). Unfortunately, most mechanical approaches are problematic or impractical, especially when a large area is to be treated, and many chemical approaches are not always well tolerated or difficult to formulate. Moreover, topically delivered dosages are often not high enough to achieve a desired effect, or when high enough, often result in systemic exposure with undesirable side effects.

Second, a drug may be orally or parenterally administered which typically allows for effective delivery. However, such administration frequently has numerous drawbacks. Most significantly, many so administered drugs are metabolized and/or excreted at a relatively high rate. Therefore, serum concentration high enough to achieve sufficient exposure of the drug to compartments such as the skin might be difficult to achieve and result in undesirable or even harmful effects.

Therefore, while numerous methods of treatment of skin related conditions are known in the art, all or almost all of them suffer from one or more disadvantages. Consequently, there is still a need to provide improved compositions and methods to improve drug delivery to the skin for treatment of skin related conditions.

### SUMMARY OF THE INVENTION

The inventors have now discovered that drugs can be safely and effectively applied to a large area of skin in relatively high concentrations without eliciting undesirable systemic effects by creating a plurality of micropores with predetermined geometry. More preferably, the pores will have a depth that is sufficient to create a channel in the stratum corneum to allow delivery of a drug to the epidermis, and more preferably to the epidermis and the dermis.

Most preferably, the majority of micropores is dimensioned such that the pore walls do not intersect with a (capillary) blood vessel. Thus, and viewed from a different perspective, it is especially preferred that the micropore walls define an area of access of a drug to the epidermal and dermal tissue layer where the amount of the drug in the systemic circulation is strongly reduced compared to injection. With respect to further micropore geometry, it should be appreciated that the side walls of micropores according to the inventive subject matter need not necessarily be straight. Indeed, it should be especially recognized that geometry of the wall of the micropores will have a significant influence on at least two factors that are critical for drug delivery: Among other things, the total inner surface can be easily modified by increasing the pore diameter and/or pore depth. Additionally, or alternatively, the wall angle may also deviate from a right angle (relative to the average surface of the stratum corneum), and as such will lead to an increase of total inner pore surface, and with that an increase in the potential area of drug delivery. Still further increases can be achieved by stepping the side walls of a pore. Second, as the micropores are subject to a healing/fill in over time, the micropore geometry will also determine the time available for delivery of a drug across the micropore walls. Third, as the micropore walls do not (or only to an insignificant degree) overlap with blood vessels, drugs applied to the micropore walls will not readily migrate into the circulation. Consequently, so applied drugs will mainly reside at high local concentration in the epidermal and dermal tissues and not rapidly transfer to the systemic circulation. Finally, micropores generated with a porator as described below will not give rise to scarring, possibly due to photoablation and/or relatively small size of the pores.

In less preferred aspects, various other techniques can be used for creating pores in biological tissues, and all known manners of creating pores are also contemplated herein. For example, suitable devices may employ heat, electric arcs and/or high-voltage pulses. U.S. Pat. No. 6,148,232, for example, disclose a technique for creating micro-channels by using an electrical field. This device could also be suitable for creating micropores of predetermined shapes, if configured to reproducibly create micropores (e.g., via feedback devices according to the inventive subject matter to detect characteristics of the individual micropores). However, microporators using a laser beam for creating pores are especially preferred.

It is further especially preferred that the laser of a laser porator is operated during pore formation in a q-switched mode and with pulse widths and energies such that laser irradiation will result in a blow-off effect without leading to coagulation. Thus, photoablation and/or photodisruption is particularly preferred. Such irradiation will typically vaporize the tissue with negligible creation of thermal damage. For example, suitable ranges of irradiance will be at least 10⁴ W/cm², and more preferably at least 10⁵ W/cm², even more preferably between 10⁵ W/cm² and 10⁹ W/cm², and most preferably between 10⁵ W/cm² and 10¹² W/cm² where energy doses of between about 0.01 J/cm² to 1000 J/cm², and more typically 0.1 J/cm² to 100 J/cm² are employed. Consequently, the laser pulse width/tissue exposure time is preferably less than 1 ms, more preferably less than 100 μs, even more preferably between 100 μs and 10 ns, and most preferably between 100 μs and 0.1 ps. Sizing and operation of lasers to achieve such parameters is well understood in the art, and many of the lasers and control systems therefore are commercially available. Consequently, and viewed from another perspective, it should be recognized that especially suitable operational parameters will be selected to provide a balance between minimum tissue damage and maximum desired effect.

However, in at least some aspects of the inventive subject matter, it may also be desirable to at least partially coagulate the pore walls A, and most typically the pore bottom 3e as exemplarily depicted in the left pore 2 in FIG. 16. The pore 2 to the left was first vertically formed in the skin 1 using q-switched laser mode or short pulsed to reduce thermal damage. The stratum corneum 1a, the epidermal layer 1b and the derma layer 1c are shown. Preferably the laser is applied several times into the same pore 2 so that the lower end 3a, 3b, 3c, 3d of the pore 2 increases with each laser pulse applied. Subsequently, the bottom 3e of the formed pore 2 is coagulated using the laser in free-running mode (using the same laser at pulse widths of 100-1000 μs), or using another laser at a wavelength and fluence effective to coagulate. Thus, laser irradiation may also include a protocol in which a pore 2 is formed using the laser in a switched mode to operate under photoablative and/or photodisruptive conditions, and in which one or more laser pulses are applied with significantly longer duration. For example, the bottom 3e of the pore 2 (and/or the side walls A where desirable) may be irradiated with the same laser or a second laser or the same laser with a second wavelength or a second laser with a second wavelength in a non q-switched or short pulsed “free-running” mode to achieve at least partial coagulation. Due to the fact that the e.g. bottom 3e of the pore 2 is sealed, the diffusion B into the epidermal layer 1b can only be achieved through the e.g. walls A of the pores 2. This leads to a decelerated delivery of the drug to the systemic circulation in the dermal layer. Moreover, such partially sealed pores 2 will provide a horizontal drug delivery (i.e., parallel to surface of skin), which may delay the rate of delivery for at least some time. Of course, over time the coagulated tissue is repaired and accelerated delivery of the drug will then take place. The person of ordinary skill in the art will readily be able to determine a suitable time for irradiation to achieve coagulation, and it is generally contemplated that appropriate pulse widths will be in the range of about 10 μs to about 1000 μs, more preferably in the range of about 100 μs to about 500 μs.

In a further aspect of the inventive subject matter, it is desirable to create a plurality of pores 2 as disclosed in FIG. 16 to the right, without coagulated pore walls A, and it is desirable to create the pores 2 with reproducible shape.

Thus, multiple applications of a drug through micropores in the same area are realized while maintaining a cosmetically and physiologically desirable environment. As applications as presented herein maintain adjacent tissue viability and structure, contemplated methods are also suitable for administration of a drug in large areas. It should further be especially appreciated that using contemplated methods, compositions, and devices will allow delivery of a drug into the skin of a patient at a dosage that would (a) otherwise generate adverse effects if systemically administered, and/or (b) otherwise not be obtainable when administered using conventional manners. Moreover, due to the control over drug delivery kinetic and dynamic via control of the pore geometry, drugs delivery can be personalized to accommodate different skin locations in a patient as well as different skin types among different patients. Similarly, drug delivery kinetic and dynamic can be tailored to a specific drug (e.g., slow delivery for fast acting drug, fast and high quantity delivery for instable drugs, etc.). Especially preferred porators, exemplary methods, and configurations are provided in the applicants' copending patent applications with the following serial numbers, all of which are incorporated by reference herein:

A micro-porator for porating a biological membrane to create a poration may be designed, for example, as the laser micro-porator disclosed in PCT patent application No. PCT/EP2006/061639 of the same applicant, and entitled “Laser microporator and method for operating a laser microporator”.

The biological membrane may be porated according to a method, for example, as disclosed in PCT patent application No. PCT/EP2005/051703 of the same applicant, and entitled “Method for creating a permeation surface”.

A micro-porator for porating a biological membrane and an integrated permeant administering system may be designed, for example, as the micro-porator disclosed in PCT patent application No. PCT/EP2005/051702 of the same applicant, and entitled “Microporator for porating a biological membrane and integrated permeant administering system”.

A system for transmembrane administration of a permeant and a method for administering a permeant may be designed, for example, as the system disclosed in PCT patent application No. PCT/EP2006/050574 of the same applicant, and entitled “A system for transmembrane administration of a permeant and method for administering a permeant”.

A transdermal delivery system for administration of a drug and a method for administering the drug may be designed, for example, as the system disclosed in PCT patent application No. PCT/EP2006/067159 of the same applicant, and entitled “Transdermal delivery system and method for treating infertility”.

Thus, in one preferred aspect of the inventive subject matter, the inventors contemplate a method of treating a skin related disease or disorder in which an area of porated skin is formed and wherein the area comprises a plurality of pores. Most typically, the area is equal or greater than 1 cm², more typically equal or greater than 10 cm², even more typically equal or greater than 25 cm², and most typically equal or greater than 100 cm². The number of pores may vary considerably, and suitable numbers include those in the range of between about 10-100,000. However, and especially where large areas are treated, higher numbers are also contemplated. Therefore, the number of pores/cm² may generally vary between about 1-10, more typically 10-100, or 100-1000, and in rare cases even higher. Similarly, the pattern of pores in the skin may vary as well, and isotropic distribution is generally preferred. However, and especially where anatomically and/or physiologically advisable, anisotropic distribution is also contemplated. For example, areas of relatively slow drug diffusion (e.g., fibrotic tissue, thick dermis, etc.) may have a higher number of pores, whereas other areas may have less. Similarly, areas with disease focus may concentrate the pores in the focus and reduce the number of pores in the periphery. Similarly, areas that require a high dosage or volume of the drug may have a higher density in pores than those that require a lower dosage.

It is generally preferred that at least some of the pores have a predetermined geometry that is at least in part a function of the drug. Moreover, the predetermined geometry will preferably control the inner pore surface area, the time to pore re-closure, and/or the pore depth (i.e., layer of epidermis or dermis that is contacted with the drug). The drug (or drugs) is then applied to the area of porated skin, which may be done in single, repeated, or continuous (e.g., under occlusion) manner. While numerous alternative wavelengths are deemed suitable, particularly preferred wavelengths for laser ablation is at a wavelength of at least 2500 nm, and most preferably at about 2950 nm.

Contemplated skin related disease or disorder are preferably those that are responsive to treatment with an immunomodulatory, immunostimulatory, or immunosuppressive drug, and particularly preferred immunomodulatory, immunostimulatory, and immunosuppressive drugs include interferons, tacrolimus, alefacept, cyclosporin A, mycophenolate, rapamycin, everolimus, glucocorticoids, infliximab, alemtuzumab, etanercept, mofetil, methotrexate, azathioprine, and ribavirin. For example, especially contemplated skin related diseases or disorders include skin graft rejection, limb (e.g., finger, hand, etc.) transplant rejection, and auto immune diseases of the skin. Thus, the term “skin related diseases or disorders” as used herein includes all diseases or disorders in which skin is diseased or has a disorder, or in which skin is involved in an at least mediatory role. For example, hand transplant rejection is thought to be mediated by lymphocytic and eosinophilic infiltration in the dermis and epidermis.

With respect to administration of contemplated drugs it is generally preferred that the total administered amount is greater than an amount given in an oral or parenteral administration, and that the drug or drugs are topically administered to the porated area in variable schedules. For example, a typical treatment schedule will include multiple porations over an extended period of time, such as once daily, weekly, or monthly over at least several days, weeks, months, and even years, wherein subsequent porations may be performed at the same, an overlapping, or different site. Similarly, and depending the particular drug, the amount administered will vary between about 1 pmol/cm² to 1 nmol/cm², or 1 nmol/cm² to 1 μmol/cm², or even 1 μmol/cm² to 1 mmol/cm² (or even higher). Where desired, application of the drug may be continuous (under occlusion or with large supply), or may be in more or more applications in form of a patch, spray, cream, emulsion, etc. The patch can be applied (e.g. after a hand transplantation) like a tape around the forearm. In case of strong side effects the patch can be removed immediately.

Therefore, in another aspect of the inventive subject matter, it is contemplated that an immunomodulatory, immunostimulatory, or immunosuppressive drug is employed in the manufacture of a medicament for treatment of a skin related disease or disorder, wherein the drug is formulated for topical administration to porated skin. Such skin will typically have a plurality of pores with predetermined geometry such that the concentration of the drug in combination with the predetermined geometry of the pores are effective in treatment of the skin related disease or disorder. For example, where mononuclear cells in dermal layers of the skin are targeted to treat rejection, suitable drugs will include tacrolimus and cyclosporin A to pores that have a depth reaching at least the epidermal layer but not the dermal layer. Depending on the desired concentration, the pores may then be formed to have a larger diameter/depth/angle (for high delivery area per pore) or a smaller diameter/depth/angle (for moderate delivery area per pore). Moreover, where larger doses are desired, the number of pores may be increased. Thus, and as discussed above, the predetermined geometry will preferably control the inner pore surface, the time to pore re-closure, and/or the delivery depth (and with that the target tissue). However, regardless of the drug delivery kinetic and dynamic, it is typically preferred that the pores have a geometry such that topical administration will not (or only to a minor degree [e.g., less than 10% of total dose, less than 5% of total dose, or less than 2% of total dose]) result in systemic delivery of the drug.

In preferred aspects, an adequate formulation of the relevant drug together with an adjusted number and geometry of pores enables for application of topical immunosuppressive compounds adjusted in dose and concentration in order to inhibit or entirely block lymphocyte infiltration into the dermis. Furthermore, it is envisaged, that the concentration of the immunosuppressant in the dermis is high enough to block chemotaxis or lymphocyte transmigration for a time of for example 2 weeks or longer. Hence, infiltration of activated T-lymphocytes as the prime mechanism of acute cellular rejection in allotransplantation shall be inhibited while avoiding systemic exposure and associated side effects.

In addition, it is envisioned that the relevant drug could be co-delivered with a conjugating substance, for example a small organic molecule, a biomolecule (e.g., proteins, polysaccharides, a deactivated virus particle), a polymeric substance or a supramolecular system such as micelles, reversed micelles, nanovesicles or liposomes, to form a conjugate, in which the relevant drug is either physically or chemically bound to the conjugating substance. This could prevent or strongly reduce the delivery of the drug into the systemic circulation by preventing passage through the tissue-blood vessel barrier or by strongly reducing the diffusion of the drug from the epidermal layer into the dermal layer.

Contemplated drugs may therefore be bundles with instructions to form a kit comprising a drug that is effective in treatment of the skin related disease or disorder, and an instruction to apply the drug to an area of porated skin that has a plurality of pores, wherein the information specifies that the area is equal or greater than 1 cm², and wherein the information also specifies that at least some of the plurality of pores have a predetermined geometry that is effective to prevent systemic administration of the drug. In especially preferred aspects, the instructions will also include one or more laser poration parameters (e.g., number, shape, density, etc, optionally as function of skin type) that determine the predetermined geometry.

Consequently, drug and medical device manufacturers will be able to facilitate treatment of a skin related disease or disorder by providing information that a drug is effective in treatment of the skin related disease or disorder. Most typically such provision is given in printed and/or displayed format, which may also include regulatory filing information for marketing such drugs and devices. Furthermore, such manufacturers may further provide information to apply the drug to an area (e.g., area is equal or greater than 1 cm²) of porated skin comprising a plurality of pores, wherein the manufacturer typically produces and/or offers at least one of the drug and the porator for sale. In a further preferred aspect, such method will also include information that further specifies that at least some of the plurality of pores have a predetermined geometry, and that the predetermined geometry is effective to prevent systemic administration of the drug. With respect to preferred diseases, drugs, devices, and further contemplations, the same considerations as provided above apply.

In still further contemplated aspects, the laser porator preferably comprises a disposable tip through which the laser beam is projected onto the skin. There are numerous such tips known in the art and all known configurations, materials, and manners of coupling are deemed suitable for use herein. However, in especially preferred aspects, the tip will also include a tissue biasing element that, upon contact with the tip, will force the tissue to be treated into a predetermined geometry. Most typically, the predetermined geometry will ensure that the average distance between the laser mirror that steers the beam over the skin and the skin that is to be treated is substantially the same. Viewed from another perspective, the tissue biasing element will deform the skin such that the skin that is to be treated will be at the focal point of the laser beam throughout the area that is to be treated. Such tissue biasing will allow for consistent application of a laser beam with known and/or uniform parameters. The following figures and description will provide sufficient guidance to a person of ordinary skill in the art to make and use the tips contemplated herein.

### BRIEF DESCRIPTION OF THE DRAWINGS

By way of example only, embodiments of the invention are described with reference to the accompanying drawings, in which:

FIG. 1 shows an exemplary longitudinal section of a tip suitable for a laser operated micro-porator;

FIG. 2 shows an exemplary surface of the tip;

FIG. 3 shows a perspective view of an exemplary tip;

FIG. 4 shows a longitudinal section of a further embodiment of a tip along (B-B);

FIG. 5 shows a cross section along (A-A) of the tip of FIG. 4;

FIG. 6 shows a longitudinal section of a further embodiment of a tip along (B-B);

FIG. 7 shows a cross section along (A-A) of the tip of FIG. 6;

FIG. 8 shows a longitudinal section of a further embodiment of a tip along (B-B);

FIG. 9 shows a cross section along (A-A) of the tip of FIG. 8;

FIG. 10 shows the front end of a longitudinal section of a further tip;

FIG. 11 shows a front view of a tissue biasing element 8a of a further tip;

FIG. 12 shows a perspective view of the tissue biasing element 8a according to FIG. 11;

FIG. 13 shows a front view of a tissue biasing element 8a of a further tip;

FIG. 14 shows a front view of a tissue biasing element 8a of a further tip;

FIG. 15 shows an intersection of the element of FIG. 11 in detail.

FIG. 16 shows a schematic cross-section of two pores of a laser porated skin.

FIG. 17 shows a laser porator;

FIG. 18 shows a cross section of a forearm with a laser micro-porator device attached;

FIGS. 19a and 19b a perspective view of examples of shapes of micro-porations;

FIG. 19c-19e show a plan view of the skin with an array of micro-porations;

FIG. 20 shows the permeation surface of all micropores over time;

FIG. 1 shows a tip 8 coupled to laser housing 9 of a laser-porator, wherein the tip is positioned proximal to the ablation site. The laser-porator comprises at least one swivel-mounted deflecting mirror 8f to deflect a laser beam 4, 4a into various directions onto the skin 1. The tip 8 forms a container with a cylindrical wall 8n and a protective glass 8i. This container collects the ablated tissue and other matter released by the ablation. The tip 8 is preferably shaped so as to allow easy attachment and removal of the tip 8 from the housing 9 of the laser-porator. The protective glass 8i is an at least partially transparent medium for the laser beam 4 and may be made of glass, polycarbonate, or another medium that is at least partial transparent for the laser beam 4. Instead of the protective glass 8i a F-Theta lense may be arranged. The use of the F-Theta lense is advantageous in conjunction with the scanning mirror 8f to create a similar pattern of the laser beam 4 on the skin 1, independent of the deflection of the scanning mirror 8f.

In one preferred embodiment, the tip 8 comprises tissue biasing element 8a which biases the portion of skin 1 that is within the opening of the tip 8 into a predetermined shape (e.g., downward, to create a bowl shape) as indicated by the line 1a. Preferably, the tissue biasing element 8a creates a concave shape having its center of curvature at point R of the deflecting mirror 8f, wherein Point R is the reflecting point of laser beam 4. Therefore, tissue biasing element 8a will allow Laser beam 4 to have about equal length between the deflecting mirror 8f and the surface 1a of the skin 1, which in turn allows to create a highly reproducible geometry of the pores within the skin.

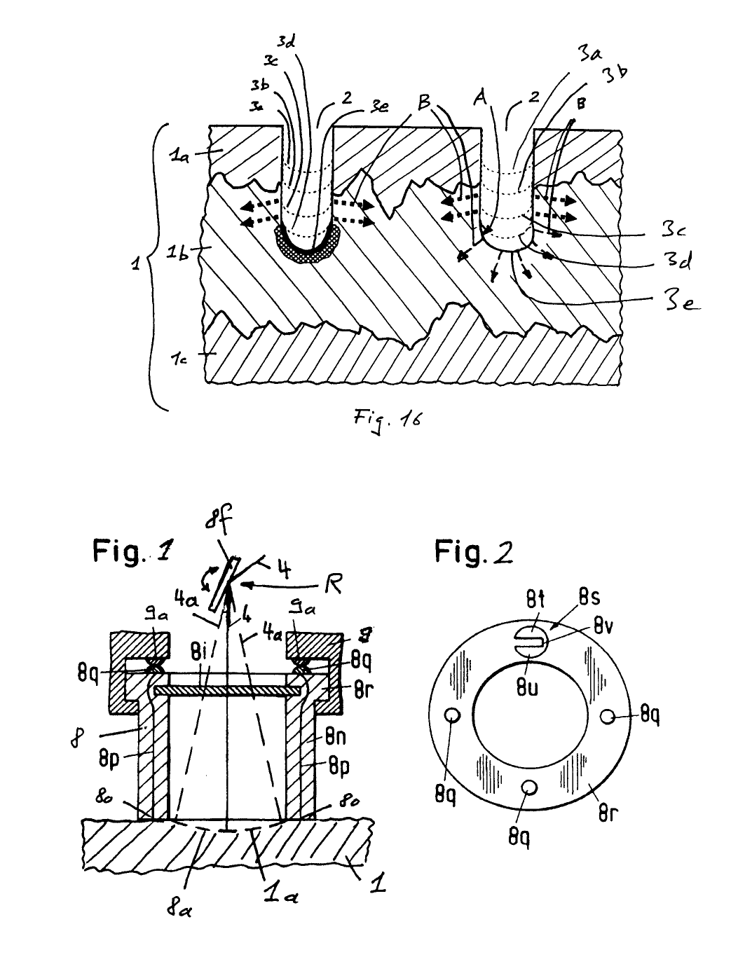

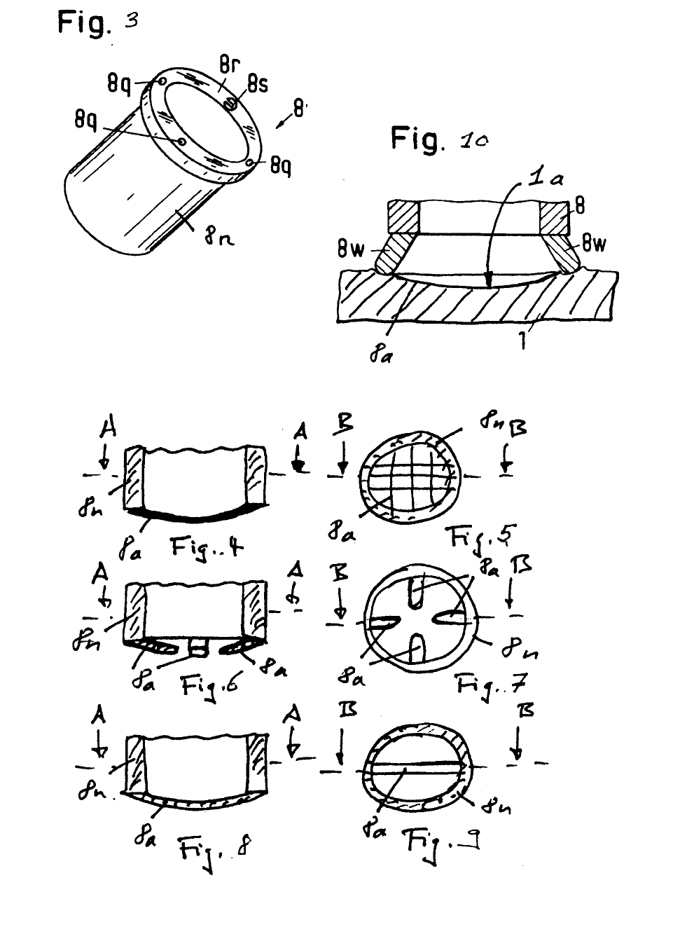

The tip 8 may further comprise electrical contact elements 8o, 8q that are electrically coupled to an electrical wire 8p. The contact elements 8q are connected with the contact elements 9a of the laser housing 9. This arrangement allows measuring various physiological parameters (e.g., impedance of the skin 1 between the contact elements 80) of the skin. Most preferably, the contacts may also be used in a locking mechanism to ensure that the tip 8 is properly positioned on the skin, before the laser source is activated. The tip 8 can comprise further sensors, for example, sensors to measure humidity, temperature, or pH of the skin. Because laser beam 4 might cause injuries if not handled properly, it is important that the laser beam 4 is only activated when the tip 8 is placed onto the skin. Thus, as shown in FIG. 2 and FIG. 3, the disposable tip 8 can include a safety mechanism 8s which allows using the tip 8 only once. The safety mechanism 8s comprises two contact elements 8t, 8u, with mating contacts in the laser housing 81, and a fusing element 8v that evaporates after a current has been applied, or breaks mechanically, or is an electronic device (e.g., a microchip), which can be reprogrammed. After poration is finished, a change is applied to the safety mechanism 8s such as burning a fuse element. The status of the safety mechanism 8s is controlled by the laser porator 10 so that the tip 8 can only be used once.

The tissue biasing element 8a of the tip 8 shown in FIG. 4 is a mesh, which may be fabricated from numerous materials, including metal, metal alloys, polymers, and all reasonable combinations thereof. The exemplary cross section illustrated in FIG. 5 shows that the wires are spaced apart to leave an intermediate space in which the laser beam 4 may irradiate the skin surface 1a. In another embodiment, the exemplary tissue biasing element 8a of the tip 8 depicted in FIG. 6 comprises four projecting pins 8a. The cross section depicted in FIG. 7 shows the four projecting pins 8a leaving an intermediate space between the projecting pins 8a as well as in the centre. Alternatively, as depicted in FIG. 8, the tissue biasing element 8a of the tip 8 comprises one projecting pin 8a. The cross section illustrated in exemplary FIG. 9 shows the projecting pins 8a leaving two intermediate spaces between the projecting pin 8a and the side wall 8n.

It is generally contemplated that the disposable tips 8 as shown in one of the FIG. 1 to 9 are removably coupled to the housing of the laser-porator. Most preferably, the laser beam 4 is triggered and deflected so that the tissue biasing element 8a is not hit by the laser beam 4 but that only the skin surface 1a is hit. In another preferred embodiment, the laser-porator comprises a detector to detect shape and orientation of the tissue biasing element 8a, to deflect and trigger the laser beam 4 such as to hit only the intermediate spaces of the tissue biasing element 8a. In such devices, the biasing element may have a identifier (e.g., bar code, reflective element, electronic circuit) that provides directly or indirectly information to the porator to identify the tip to the porator.

The tip 8 may further comprise one or more elements 8w to stretch the skin 1, for example, an elastic ring as shown in FIG. 10. When the tip 8 is pressed onto the skin 1, the elastic ring pushed the skin 1 outward in radial direction, so that the skin within the area enclosed by the elastic ring is stretched. Thus, the surface of the skin is pulled tight on the tissue biasing element 8a.

FIG. 11 shows a front view and FIG. 12 a perspective view of a further tissue biasing element 8a having the shape of a partly hemispherical mesh, and comprising elastic material (e.g., metallic wire or plastic element), or comprising a rigid material (e.g., metal). The tissue biasing element 8a includes metallic wires 8b connected with an outer ring 8c (preferably made from the same material as the element 8b). As appropriate, the outer ring 8c may be attachable to the cylindrical wall 8n, or may be integral part of the tip 8.

FIG. 13 shows a front view of an exemplary tissue biasing element 8a leaving an intermediate space in its center. FIG. 14 shows a front view of an exemplary tissue biasing element 8a having two sensors 8d arranged on the outer ring 8c (the sensors 8d may be electrically coupled via wires 8p).

It should further be appreciated that various types of connectors (e.g., snap lock or threaded) are suitable to coupled the tip 8 with the housing 9. In one preferred embodiment, the position of the tip 8 with respect to the housing 9 is checked before the laser-porator is directed onto the skin. In another preferred embodiment, the tip 8 comprises an indicator 8f which allows detecting the position of the tip 8 with respect to the housing 9. The indicator 8f can be a reflective surface on the tissue biasing element 8a, which may be arranged on the cross section 8e of the elements 8b as illustrated in FIG. 15. The orientation of the indicator 8f can be detected with a sensor or with the laser beam 4 in combination with a sensor. The indicator 8f may be a reflective area. In a further preferred embodiment, the indicator 8f may be used as safety mechanism 8s in which the properties of the indicator 8f are altered when the tip 8 is used. For example, the laser beam 4 may be directed onto the indicator 8f after porating the skin, to alter or destroy a small reflective layer forming the indicator 8f. Before starting porating the skin, a controller of the laser porator may, by using the laser beam 4 or another sensor, check the status of the indicator 8f, and depending on properties of the indicator 8f, permit or deny poration.

FIG. 17 shows a laser micro-porator 10 comprising a Q-switched or short pulsed laser source 7 and a laser beam shaping and guiding device 17. The laser source 7 has a light source 7c for optical excitation of a laser active material 7b, and a set of reflecting mirrors 7d,7e. The laser source 7 comprises a laser cavity 7a containing a laser crystal 7b, preferably Er and optional additionally Pr doped YAG, which is pumped by an exciter 7c, the exciter 7c being a single emitter laser diode or a set of single emitter laser diode arrays like emitter bars or stacks of emitter bars. The laser source 7 further comprising an optical resonator comprised of a high reflectance mirror 7d positioned posterior to the laser crystal 7b and an output coupling mirror 7e positioned anterior to the laser crystal 7b, and a saturable absorber 7f positioned posterior to the laser crystal. The saturable absorber 7f works as a Q-switch. A focusing lens 17a and a diverging lens 17b are positioned beyond the output coupling mirror 7e, to create a parallel or quasi-parallel laser beam 4 or a focused laser beam 4. Instead of the lenses 17a, 17b, the microporator 10 could comprise different optical means 17a, 17b, which, for example, focus the laser beam 4 onto the surface of the skin 1. The diverging lens 17b can be moved by a motor 17c in the indicated direction. This allows a broadening or narrowing of the laser beam 4, which allows changing the width of the laser beam 4 and the energy fluence of the laser beam 4. A variable absorber 17d, driven by a motor 17e, is positioned beyond the diverging lens 17b, to vary the energy fluence of the laser beam 4. A deflector 8f, a mirror, driven by an x-y-drive 8g, is positioned beyond the absorber 17d for directing the laser beam 4 in various directions, to create individual pores 2 on the skin 1 on different positions. A control device 11 is connected by wires 1a with the laser source 7, drive elements 17c, 17e, 8g, sensors and other elements not disclosed in detail.

In a preferred embodiment the laser porator 10 also includes a feedback loop 13 respectively a feedback mechanism. In FIG. 17, the feedback loop 13 comprises an apparatus 9 to measure the depth of the individual pore 2, and preferably includes a sender 9a with optics that produce a laser beam 9d, and a receiver with optics 9b. The laser beam 9d has a smaller width than the diameter of the individual pore 2, for example five times smaller, so that the laser beam 9d can reach the lower end of the individual pore 2. The deflection mirror 8f directs the beam of the sender 9a to the individual pore 2 to be measured, and guides the reflected beam 9d back to the receiver 9b. This distance measurement device 9, which can be built in different way, allows measuring the position of the lower end e.g. the depth of the individual pore 2. In a preferred embodiment, the depth of the individual pore 2 is measured each time after a pulsed laser beam 4 has been emitted to the individual pore 2, allowing controlling the effect of each laser pulse onto the depth of the individual pore 2. The feedback loop 13 can be built in various ways to be able to measure a feedback signal of an individual pore 2. The feedback loop 13 may, for example, comprise a sender 9a and a receiver 9b, built as a spectrograph 14, to detect changes in the spectrum of the light reflected by the lower end of the individual pore 2. This allows, for example, detecting whether the actual lower end 3a, 3b, 3c, 3d of the individual pore 2 is part of the stratum corneum 1a or of the epidermis 1b. The laser porator 10 also comprises a poration memory 12 containing specific data of the individual pores 2, in particular the initial microporation dataset. The laser porator 10 preferably creates the individual pores 2 as predescribed in the poration memory 12. The laser porator 10 also comprises one or more input-output device 15 or interfaces 15, to enable data exchange with the porator 10, in particular to enable the transfer of the parameters of the individual pores 2, the initial microporation dataset, into the poration memory 12, or to get data such as the actual depth or the total surface Ai of a specific individual pore 2i. The input-output device 15 can be a card reader, a scanner, a wired interface or for example a wireless connection such as Bluetooth.

The porator further can comprise one or more input-output devices or user interfaces 15 for manually exchange date like data of substances, individuals and much more. The user interface can for example comprise displays, buttons, voice control or a finger print sensor.

There are different ways to build a laser source 7. The laser source 7 may, for example, be built as a laser diode with optics that create a beam 4 of fixed width, for example a width of 250 μm.

The pulse repetition frequency of the laser source 7 is within a range of 1 Hz to 1 MHz, preferably within 100 Hz to 100 kHz, and most preferred within 500 Hz to 10 kHz. Within one application of the laser porator 10, between 2 and 1 million individual pores 2 can be produced in the biological membrane 1, preferably 10 to 10000 individual pores 2, and most preferred 10 to 1000 individual pores 2, each pore 2 having a width in the range between 0.05 mm and 0.5 mm or up to 1 mm, and each pore 2 having a depth in the range between 5 μm and 200 μm, but the lower end of the individual pore 2 being preferably within the epidermis 1b. If necessary the porator 10 is also able to create pores of more than 200 μm depth.

The laser porator 10 also comprises an interlock mechanism, so that a laser pulse is emitted only when it is directed onto the skin 1. The feedback loop 13 could for example be used to detect whether the pulse is directed onto the skin 1. Those skilled in the art will appreciate that there are numerous ways to create an interlock mechanism, and all such ways are contemplated. One embodiment is described in FIG. 4a.

FIG. 17 discloses a circular laser beam 4 creating a cylindrical individual pore 2. The individual pore 2 can have other shapes, for example in that the laser beam 4 has not a circular but an elliptical shape, a square or a rectangle. The individual pore 2 can also be shaped by an appropriate movement of the deflector 8f, which allows creation of individual pores 2 with a wide variety of shapes.

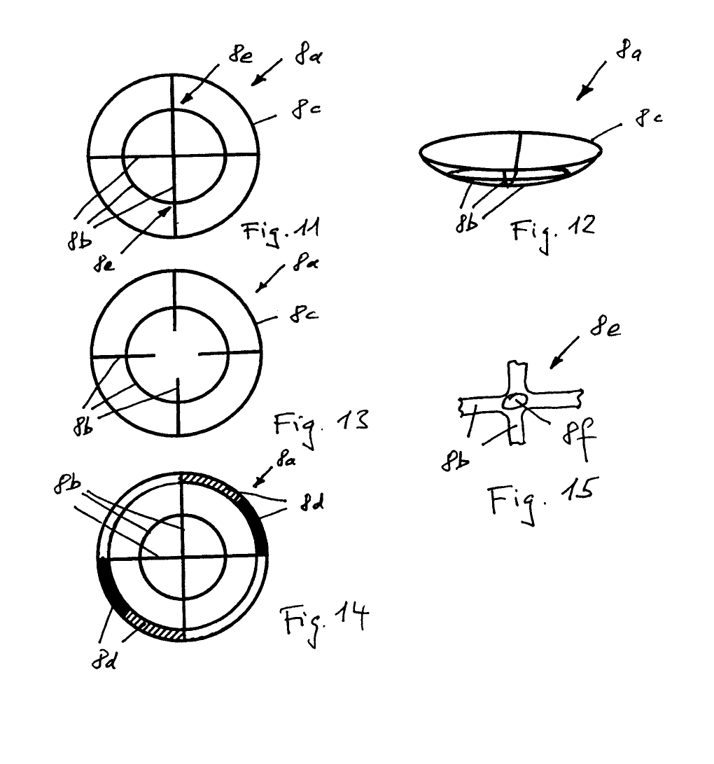

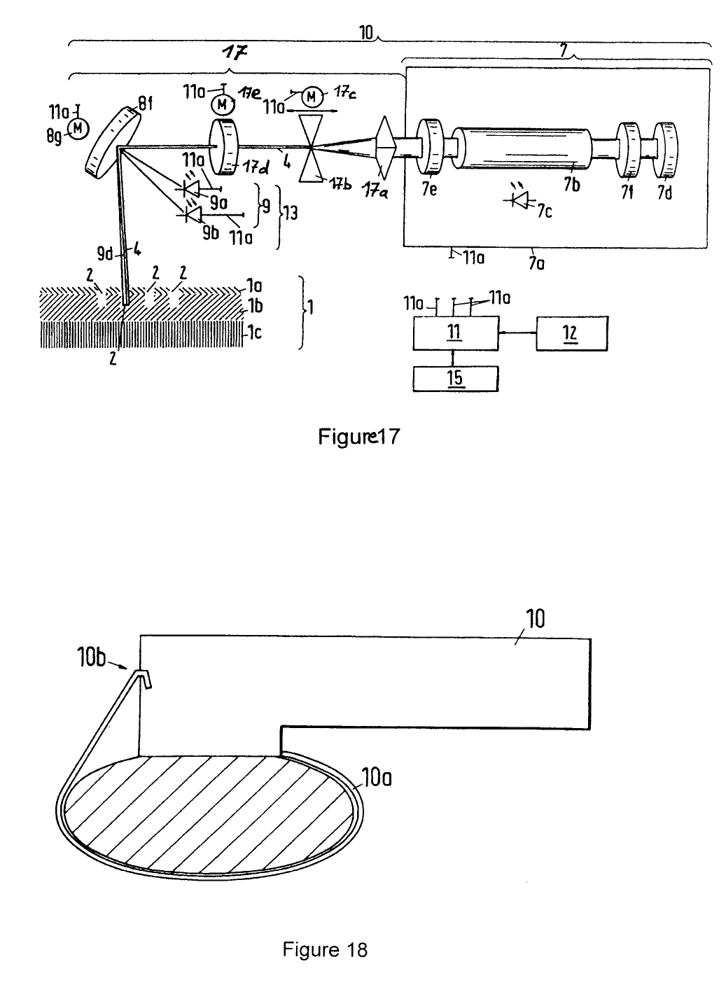

FIG. 18 shows a cross-section of a forearm. A laser micro-porator device 10 may releasable be attached to the forearm using an elastic belt 10a comprising a connector 10b. This attachment allows suppressing or reducing a relative movement between the micro-porator 10 and the area of the forearm on which the front part of the micro-porator 10 is arranged. To porate a skin area of equal or greater than 1 cm² it might be useful to release the elastic belt 10a after porating, to remove the ablator 10 relative to the skin, and to again fix the elastic belt 10a, so that the ablator 10 may porate an additional area of the skin. Thus allowing to porate a large area of 25 cm², 100 cm² or even more. The ablator 10 may also be used without fixing it's position by repeatedly pressing the tip of the ablator onto the skin area to be porated, and by then activating the ablator. The ablator 10 may also be mounted on a scanner which is able to position and to control position of the ablator 10 with respect to the skin, thus allowing a very accurate and controllable poration of a large area of the skin.

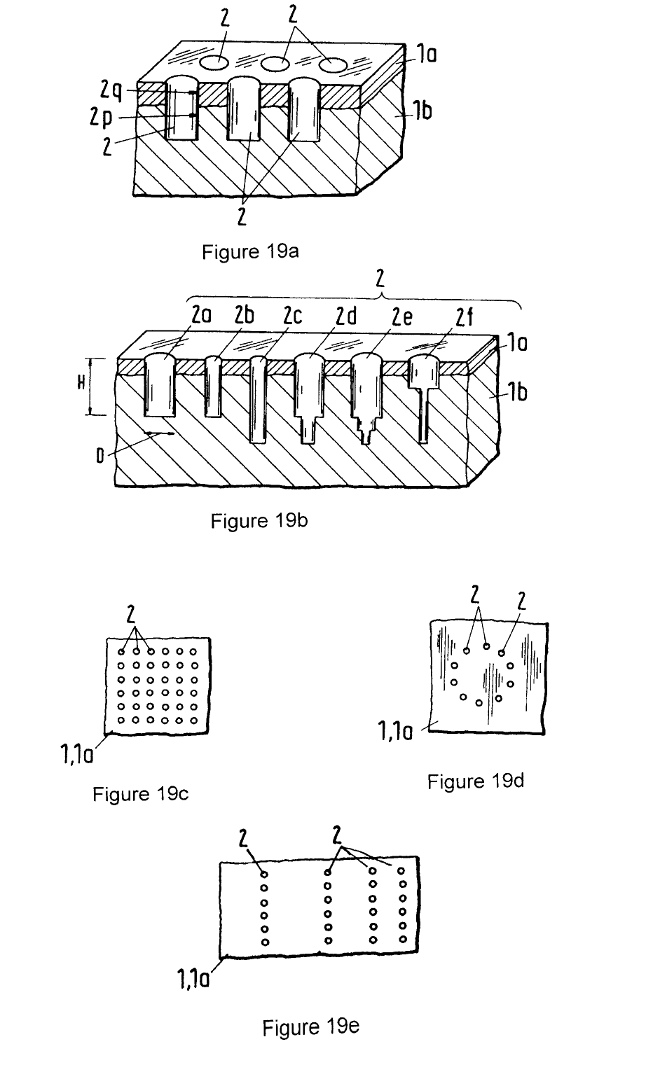

FIG. 19a shows an array of individual pores 2 in the skin 1. All individual pores 2 have about the same shape and depth.

FIG. 19b shows individual pores 2a to 2f of various shapes, which can be created with support of the poration controller 11 controlling the laser porator 10. To produce the individual pores shown in FIG. 19b, at least the cross-section of the laser beam 4 has to be varied. In a preferred embodiment, the laser porator 10 varies the cross-section and/or the energy density of each consecutive pulsed laser beam 4, which allows creation of individual pores 2 with numerous different shapes.

FIG. 19c shows a plan view of the skin having a regular array of individual pores 2 that collectively form a micro-poration. The micro-poration on the biological membrane, after the laser porator 10 has finished porating all pores, is called “initial microporation”. The poration memory 12 preferably contains the initial microporation dataset, which define the initial microporation. The initial microporation dataset comprises any suitable parameters, including: width, depth and shape of each pore, total number of individual pores 2, geometrical arrangement of the pores 2 on the biological membrane, minimal distance between the pores 2, locations on the skin the porator has to porate the skin, and so forth. The laser porator 10 creates the pores 2 as defined by the initial microporation dataset. This also allows arranging the individual pores 2 in various shapes on the skin 1, as for example disclosed with FIG. 19 d. FIG. 19e discloses a further advantageous arrangement of the pores in the skin. The density of the number of pores 2 is increasing or decreasing. This allows a certain area in the skin to be supplied with a larger or smaller amount of drug.

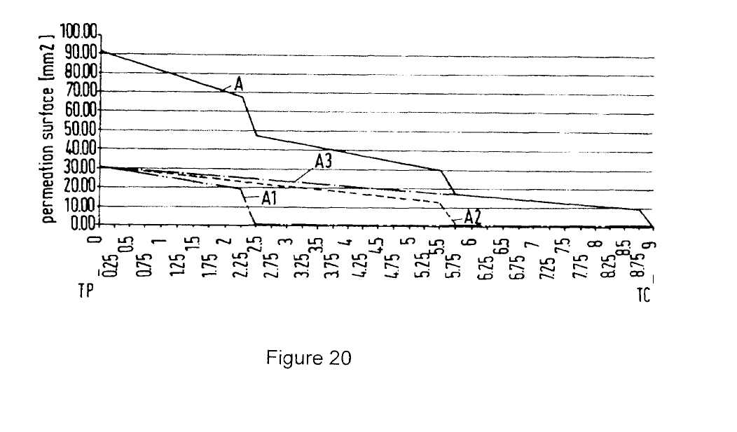

FIG. 20 shows an example of the total permeation surface A as a function of time. The laser-porator 10 allows to micro-porating the tissue such as the skin 1 by the creation of an array of micropores 2 in said tissue 1, whereby the number of micropores 2 and the shape of these micropores 2 is properly selected so that the sum of the micropores 2 forming an initial permeation surface, and that the permeation surface A (t) of the initial permeation surface decreases in a predetermined function over time, due to cell growth in the micropores 2.

The initial microporation dataset according to FIG. 20 comprises three groups of cylindrical micropores 2 with different shapes:

a first group consisting of 415 pores with a diameter of 250 μm, a depth of 50 μm and a permeation surface A1 as a function of time.

a second group consisting of 270 pores with a diameter of 250 μm, a depth of 100 μm and a permeation surface A2 as a function of time.

a third group consisting of 200 pores with a diameter of 250 μm, a depth of 150 μm and a permeation surface A3 as a function of time.

The total permeation surface A as a function of time is the sum of all three permeation surfaces A1, A2 and A3.

All individual pores 2i, which means the initial microporation, is created within a very short period of time, for example, within a time range of a fraction to a few seconds up to a few seconds or a view minutes, so that beginning with the time of poration TP, the sum of all created pores 2i forming an initial permeation surface, which, due to cell growth, decreases as a function of time. At the time TC all individual pores 2i are closed, which means that the barrier properties significantly increase.

### CONTEMPLATED EXAMPLES

### Treatment of Rejection

In one non-limiting example, it is contemplated that a patient receives after bilateral hand transplantation alemtuzumab (2×20 mg) for induction therapy. Prophylactic immunosuppression preferably includes administration of tacrolimus, mycophenolate mofetil, and steroids, typically at an initial dosage of 500 mg with a rapid taper to 5 mg maintenance therapy. Tacrolimus is administered at two daily oral doses to achieve trough levels of 15-20 ng/ml early after transplantation and is subsequently tapered to 10 ng/ml.

The patient is then monitored for signs and symptoms of rejection and treatment modified as appropriate. For example, a first rejection episode may be observed at two months after transplantation, with lesions being restricted to the dorsal side of the hand and spreading over an area of approximately 12-15 cm². Conventional treatment would typically employ systemic administration of bolused steroids together with an increase of maintenance immunosuppression for treatment of rejection. In contrast, the inventors now contemplate that topical application of e.g. tacrolimus or alemtuzmab to porated skin may replace or complement conventional therapy. In such treatments, it should be appreciated that the drugs will be rapidly available to the site of action and may therefore present an effective route with significantly reduced potential adverse effects. So administered drugs may be applied to the porated skin under various schedules, including once daily (or less) to several times a day at constant, increasing, or decreasing dosages. With respect to appropriate dosages, the same considerations as provided above apply.

### Prophylaxis of Rejection

Immunosuppression after e.g. hand or face transplantation could be follows: Induction therapy with alemtuzumab or anti-thymozyte globulin is followed by tacrolimus monotherapy aiming for trough levels of as low as 5-8 ng/ml. In addition, the skin of the transplant is prepared for drug uptake by creating porated areas of the skin scattered on the transplant at a distance of abut 3-5 cm in all directions. Subsequent application of suitable drugs (e.g., efomycin) onto the porated areas of the skin is preferably in an amount effective to inhibit lymphocyte infiltration in the dermis to thereby prevent rejection. Topical treatment of the skin is repeated as needed (e.g., every three weeks during the first six months continued by application at intervals of 6 weeks until the end of the first year). Thereafter the regimen of topical treatment is continued at predefined intervals.

### Treatment of Psoriasis

Psoriatic lesions can be restricted to small areas of the skin or spread over large areas with often debilitating effect on a patient's daily activities. Systemic therapies are limited by drug toxicity but recently, treatment with efalizumab has been shown to attenuate the disease in 22-28% of patients. It is likely that limitations of efficacy of efalizumab treatment are caused by inadequate concentrations and/or distribution to all affected skin areas. Consequently, the inventors therefore contemplate that preparation of all affected skin areas by creating porated areas prior to topical application of efalizumab or a comparable inhibitor of lymphocyte migration might substantially improve the efficacy of such a therapeutic approach.

The following list discloses by example a list of drugs suitable to be used in combination with the laser porator, in particular for wide-area treatment of skin related conditions.

TABLE 1
Examples of suitable drugs

| Generic name | Brand name | Type of drug |
|---|---|---|
| Lymphozyte | Atgam | |
| immuno globulin | | |
| 6- mercaptopurine | Purinethol | antimetabolites |
| Abciximab | ReoPro | monoclonal antibody |
| Adalimumab | Humira | monoclonal antibody |
| Alemtuzumab | Campath-1H | monoclonal antibody |
| Anti thymozyte- | Thymoglobulin | Immuno suppressive |
| globulin | | agent |
| Azathioprine | Imuran/Immunoprin | Immuno suppressant |
| Basiliximab | Simulect | monoclonal antibody |
| Betamethasone | Betnesol/Betnelan | Corticosteroids |
| Bevacizumab | Avastin | monoclonal antibody |
| Cetuximab | Erbitux | monoclonal antibody |
| Cyclophosphamine | | |
| Cyclophosphamide | Cytoxan/Neosar | |
| Cyclosporine A | Gengraf/Neoral/SangCya; Sandimmune | Immuno suppressant |
| Daclizumab | Zenapax | monoclonal antibody |
| Deflazacort | Calcort | Corticosteroids |
| Dexamethasone | Dexsol | Corticosteroids |
| Etanercept | Enbrel | |
| Everolimus | Certican | Immuno suppressant |
| Fingolimod | | Immuno modulator |
| Hydrocortisone | Efcortesol/Hydrocortistab/Solu-cortef/ | Corticosteroids |
| Hydroxyurea | | antimetabolites |
| Infliximab | Remicade | monoclonal antibody |
| Interferon-alpha | | |
| Interferon-beta | | |
| Lacquinimod | | Immuno modulator |
| methotrexate | Rheumatrex/Trexall | antimetabolites |
| Methylprednisolone | Solu-medrone/Medrone/Depo-medrone/ | Corticosteroids |
| | Medrol | |
| Muromonab-CD3 | OKT3 | monoclonal antibody |
| Mycophenolate- | CellCept | Cell cyclus inhibitor |
| mofetil | | |
| Mycophenolic Acid | Myofortic | |
| Nilotinib | Tasigna | monoclonal antibody |
| Omalizumab | Xolair | monoclonal antibody |
| Opioids | | |
| Prednisolone | Precortisyl/Deltastab/Deltacortil | Corticosteroids |
| Prednisone | Deltasone, Orasone, Prednicen-M, Liquid Pred | Corticosteroids |
| Rituximab | Rituxan | monoclonal antibody |
| Sirolimus/ | Rapamycin, Rapamune | Protein kinase |
| Rapamycin | | inhibitors |
| Tacrolimus | Prograf, Protopic, Advagraf | Immuno suppressant |
| Tositumomab | Bexxar | monoclonal antibody |
| Trastuzumab | Herceptin | monoclonal antibody |
| Triamcinolone | Kenalog | Corticosteroids |

Thus, specific embodiments and applications of drug delivery to skin for treatment of skin related conditions have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

It should be especially appreciated that the tip 8 described herein is configured to be used in combination with a laser porator. Therefore, it should be recognized that such tips may not only be used for treating skin related conditions but may also be used independently in applications where microporation, and particularly microporation with predetermined pore geometry or drug delivery kinetic/dynamic is required. For example, contemplated alternative uses include application of the tip to create pores for systemic, transdermal administration of permeants and drugs, such as the administration of high amount of drugs, and also the transdermal administration through an area of equal or less than 1 cm².

1. A method of treating a skin related disease or skin related disorder comprising:
topically applying to a porated skin a formulation for topical administration wherein the formulation comprises at a concentration an immunomodulatory, immunostimulatory, or immunosuppressive drug;
wherein the porated skin has a plurality of pores with predetermined geometry such that the concentration of the drug in combination with a predetermined geometry of the plurality of pores are effective in treatment of the skin related disease or skin related disorder.

2. The method of claim 1 wherein the skin related disease or disorder is a skin graft rejection, a disease or disorder in which skin is involved in an at least mediatory role, a limb transplant rejection, or an autoimmune disease.

3. The method of claim 1 wherein the predetermined geometry controls at least one of inner pore surface, diameter of the pore, time to pore re-closure, and determination of delivery depth.

4. The method of claim 1 wherein the plurality of pores have a geometry such that topical administration will not result in or strongly reduce the systemic delivery of the drug.

5. The method of claim 1 wherein the drug is conjugated to a moiety that reduces or prevents entry of the drug into systemic circulation.

6. A method of using a porator to create an area of porated skin of equal or greater than 1 cm², for a wide area administration of a drug through the skin.

7. The method of claim 6, wherein the porator is a laser porator adapted to direct a pulsed laser beam onto the skin to create a plurality of pores, and where the laser porator is adapted to hit at least a single one of the plurality of pores at least twice.

8. A method of facilitating treatment of a skin related disease or disorder, comprising:
providing information that a drug is effective in treatment of the skin related disease or disorder;
providing information to apply the drug to an area of porated skin comprising a plurality of pores;
wherein the information specifies that the area is equal or greater than 1 cm²;
wherein the information further specifies that at least some of the plurality of pores have a predetermined geometry; and
wherein the predetermined geometry is effective to substantially prevent systemic administration of the drug.

9. The method of claim 8 wherein the skin related disease or disorder is responsive to treatment with an immunomodulatory, immunostimulatory, or immunosuppressive drug.

10. The method of claim 8 wherein the skin related disease or disorder is a skin graft rejection or limb transplant rejection.

11. The method of claim 8 wherein the skin related disease or disorder is an autoimmune disease, an allergic reaction, or a disease or disorder in which skin is involved in an at least mediatory role.

12. The method of claim 8 wherein the drug is selected from the group consisting of an immunomodulatory drug, an immunostimulatory drug, an immunosuppressive drug, an anti-inflammatory drug, and a steroidal drug, optionally conjugated to a moiety that reduces or prevents entry of the drug into systemic circulation.

13. The method of claim 8 wherein the area is between 1 cm² and 25 cm², or between 25 cm² and 100 cm².

14. The method of claim 8 wherein the pores are formed in a manner effective to reduce or prevent scar formation.

15. The method of claim 8 wherein the number of pores in the area of porated skin is between 10 and 100,000.

16. The method of claim 8 wherein the predetermined geometry is at least in part a function of the drug.

17. The method of claim 8 wherein the predetermined geometry comprises at least one of determination of inner pore surface, determination of time to pore re-closure, pore depth, and determination of delivery depth.

18. A kit comprising: (a) a drug that is effective in treatment of the skin related disease or skin related disorder, and (b) an instruction to apply the drug to an area of porated skin comprising a plurality of pores, wherein the information specifies that the area is equal or greater than 1 cm², and wherein the information further specifies that at least some of the plurality of pores have a predetermined geometry that is effective to prevent systemic administration of the drug.

19. The kit of claim 18 wherein the skin related disease or disorder is responsive to treatment with an immunomodulatory, immunostimulatory, or immunosuppressive drug.

20. The kit of claim 18 wherein the skin related disease or skin related disorder is a skin graft rejection, a disease or disorder in which skin is involved in an at least mediatory role, a limb transplant rejection, or an autoimmune disease.

21. The kit of claim 18 wherein the instruction further provides laser poration parameter that determine the predetermined geometry, and wherein the pores are formed in a manner to reduce or prevent scar formation.

22. A method of treating a skin related disease or skin related disorder, comprising:
forming an area of porated skin comprising a plurality of pores, wherein the area is equal or greater than 1 cm², and wherein at least some of the plurality of pores have a predetermined geometry;
wherein the predetermined geometry is at least in part a function of the drug and controls at least one of inner pore surface, time to pore re-closure, and delivery depth; and
applying the drug to the area of porated skin.

23. The method of claim 22 wherein the step of forming the area of porated skin comprises laser ablation at a wavelength of at least 2500 nm.

24. The method of claim 22 wherein the skin related disease or skin related disorder is responsive to treatment with an immunomodulatory, immunostimulatory, or immunosuppressive drug.

25. The method of claim 22 wherein the skin related disease or skin related disorder is a skin graft rejection, a disease or disorder in which skin is involved in an at least mediatory role, limb transplant rejection, or a skin autoimmune disease.
